(19) United States
(12) Patent Application Publication
(10) Pub. No.: US 2010/0297601 A1
(43) Pub. Date: Nov. 25, 2010

(54) Small volume and ultra speed colorimetric sensor

(76) Inventor: Tom Cheng Xu, Castro Valley, CA (US)

Correspondence Address: Tom Xu, 21010 Sherman Dr., Castro Valley, CA, 94552, US

(21) Appl. No.: 12/454,639

(22) Filed: May 21, 2009

(51) Int. Cl. C12Q 1/00

(52) U.S. Cl. 435/4

(57) ABSTRACT

A sensor designed to accommodate an aqueous analyte sample having a volume of less than 1 μL and can be used to quantify the amount and concentration of such analyte through light reflectance or fiber-optic light reflectance. The sensor includes a reaction membrane and a fixed-volume sample chamber. The fixed-volume sample chamber acts to collect the sample and to secure such sample while it undergoes detection. The reaction membrane contains all of the chemicals and enzymes needed to cause a color reaction when contacted with the sample. The amount of analyte can be determined by light reflectance intensity. The sensor can be used with a meter for in-vitro clinical diagnosis and for patient self-monitoring of a physical condition, such as blood glucose levels.

### BACKGROUND OF THE INVENTION

1. Field of the Invention

The present invention relates to analytical sensors; more specifically, to sensors configured to accommodate submicroliter sample volumes in the detection of analytes in such samples.

2. Description of the Prior Art

Portable analyte monitoring devices, such as portable blood glucose monitors, typically require sensors for the quantification of analytes in biological samples. These sensors are designed to receive fluid samples from their users and are oftentimes discarded after each use. The frequent purchase and use of such sensors has become an indispensible part of the lives of many diabetics. The American Diabetes Association recommends that individuals with Type 1 diabetes measure their blood glucose level three or more times per day. Many diabetics falter from their suggested monitoring schedules. The prior art has undertaken to achieve this by operating on less blood from their users and favoring design features that lower the cost of such sensors. Features that lead to more rapid test results can also result in more frequent self-monitoring.

U.S. Pat. Nos. 4,935,346, 5,049,487, 5,059,394, 5,179,005 and 5,304,468 to Phillips et al. discloses various methods and devices for applying a sample of whole blood to the “sample” side of a test strip membrane that is impregnated with the necessary reagents. Red blood cells are then separated by this membrane as the remaining sample migrates toward the “testing” side of the membrane. The glucose in the remaining sample then interacts with the reagents to produce a light-absorbing reaction product. An optical meter can then be used to measure the color change which correlates to the blood glucose level in the sample. U.S. Pat No. 5,972,294 to Smith et al. discloses a reagent sensor dependent on a membrane for receiving a fluid sample and separating red blood cells from such a sample before an assay is performed. U.S. Pat. Nos. 5,296,192 and 6,040,195 to Carroll et al. describe an improved multi-layered sensor for receiving a whole blood sample. The test strip includes filtration layers to remove red blood cells, fluid volume control dams to prevent spillage of the fluid from the strip, and a chemical reagent formulation that facilitates end-point testing. U.S. Pat. No. 6,924,093 to Haviland et al. discloses an alignment notch added to a sensor and an obround-shaped aperture for receiving fluid samples. Both features are intended to reduce the amount of a fluid sample needed to provide an assay. Haviland indicates that the purpose of the alignment notch is to facilitate proper alignment of the sensor within a measuring instrument such that the sample-receiving aperture is accurately aligned over the instrument's light source. Prior to such an improvement, sensors compensated for the likelihood of misalignment by providing a larger measurement area in the form of a larger aperture. Such an aperture naturally required a greater sample volume to saturate.

While the above test strips and methods are all widely used, they share some common limitations. First, since blood from a diabetic patient must be applied to either a top “sample” layer or a top oval aperture of the aforementioned test strips, 3 μL to 50 μL of blood must be obtained from a patient using such a test strip. A patient must often obtain any sample volume larger than 3 μL by lancing the skin on his or her fingertips and, subsequently, milking the area to obtain a useful sample volume. This procedure is a nuisance for the patient and may often be painful. Less painful methods for obtaining a sample include lancing the arm or thigh, both of which have a lower nerve ending density than fingertips. However, lancing the body in such regions often produces inadequate sample volumes because these regions are not heavily inundated with the necessary capillary vessels. In addition, most current test strips are not designed to accept blood from these parts of the body. Although U.S. Pat. No. 6,099,484 to Douglas et al. has aimed to solve this problem by using a capillary device as a wick to transfer fluid samples to the strip pad, adding the capillary device disclosed by Douglas to most test strips would be impractical and increase the cost of such test strips prohibitively.

Douglas et al. in U.S. Pat. No. 6,099,484 discloses a test strip affixed to an end of a capillary tube for receiving fluid samples. An absorbent pad is disposed between the test strip and capillary tube for spreading-out the fluid being transferred to the test strip. An on-site analyzer such as an optical analyzer and/or an electrochemical analyzer can be mounted in the device for analyzing the fluid.

Second, since the analyte of interest is measured by the light signal reflected off the surface of a test strip where a color reaction has taken place, the strip has to be inserted into the shroud of the meter during testing to avoid interference from environmental lights. This also requires that the surface of the test strip be closely placed near the light source and the light detector of the meter. Repeated testing could potentially result in contamination of the meter by blood or other biological fluids and lead to inaccurate test results.

Most of the current test strips are designed for using fingertip blood samples, unsuitable for either arm or thigh blood because of the restricted accessibility from these parts of the body to the sample area on the strip.

Third, another desired feature for a self-monitoring system is to get the results fast, for example, within a few seconds. Diabetic patients normally take their blood sugar test before each meal. Obtaining the test results quickly is always desired, especially for children patients.

To overcome these limitations, it is desirable to develop a testing system that requires a minimal sample volume, eliminates contamination to meters and is capable of using not only fingertip blood samples but also blood samples from other parts of body, such as the arm and thigh, which have a lower nerve ending density making the sampling process less painful or even painless.

Over the past two decades, a wide variety of optical chemical sensors have been proposed for analysis of chemical species in industrial, environmental and biological samples. These sensors operate by detecting optical changes of a sensing material or indicator dye on interaction with an analyte. Due to the variety of analyte-specific indicators available, such sensors may be used for monitoring a large number of analytes, including blood glucose testing for diabetics.

For example, U.S. Pat. No. 5,859,937 to Nomura disclosed a sensor comprising an atomic oxygen etched optical fiber with analyte-responsive reagents deposited on the etched surface. The analyte concentration was measured by physical or chemical response upon being contacted with the reagents. However, optical fiber surface etching described in the patent is not practical for making reproducible and reliable test sensors.

Raskas in U.S. Pat. No. 6,157,442 described a micro optical fiber sensor device, but it is only for in vivo use.

The applicant in U.S. application Ser. No. 10/038,263 has disclosed a disposable optical fiber test tip sensor system that is easy to make and simple to use for analyzing biological samples using less than 1 microliter blood sample.

The disclosures of the above patents are incorporated herein by reference.

The current invention discloses new reflectance micro optical sensors that use ultra small sample volume for analyte detection and quantitative determination within 3 seconds of time. The new invention also discloses a sample dosage mechanism to make the test results more accurate and reliable for optical sensors.

### SUMMARY OF THE INVENTION

The optical micro-sensors of the current invention provide a method for the detection and quantification of analytes for samples in ultra small volume in ultra speed time. A micro-sensor of the present invention utilizes a very simple peroxidase-mediated colorimetric reaction. To increase the test accuracy and precision, a sample dosage mechanism is introduced to the sensors. The present invention is particularly suitable for use with an optical analyzing mechanism comprised of a light source and a light sensor.

One embodiment of the invention includes a method for determining the concentration of an analyte in a sample by contacting the sample with an optical sensor to cause a color change and then determining the concentration of the analyte. The optical sensor includes a membrane coated with coloring chemicals and enzymes. The coated membrane is sandwiched between two thin plastic films by adhesives with two open ends.

The invention is a method of determining the concentration of an analyte in a sample by light reflectance. In this method, a liquid sample is applied to one side of the membrane of the micro sensors and the color change is measured from the other side. The analyte concentration is determined by the reflected light intensity.

These and other features of the invention will become apparent to those skilled in the art upon reviewing the drawings and accompanying descriptions of the current invention presented below.

### BRIEF DESCRIPTION OF THE DRAWINGS

Referring now to the drawings:

FIG. 1 is a schematic view of the structure layout of a reflectance optical micro sensor in accordance with the principles of the present invention;

FIG. 2 is a top view of an embodiment of a reflectance sensor in accordance with the principles of the present invention;

FIG. 3 is a graph of the correlation between light reflectance intensity and blood glucose concentration using the sensor of FIG. 1.

### DETAILED DESCRIPTION OF THE PREFERRED EMBODIMENT

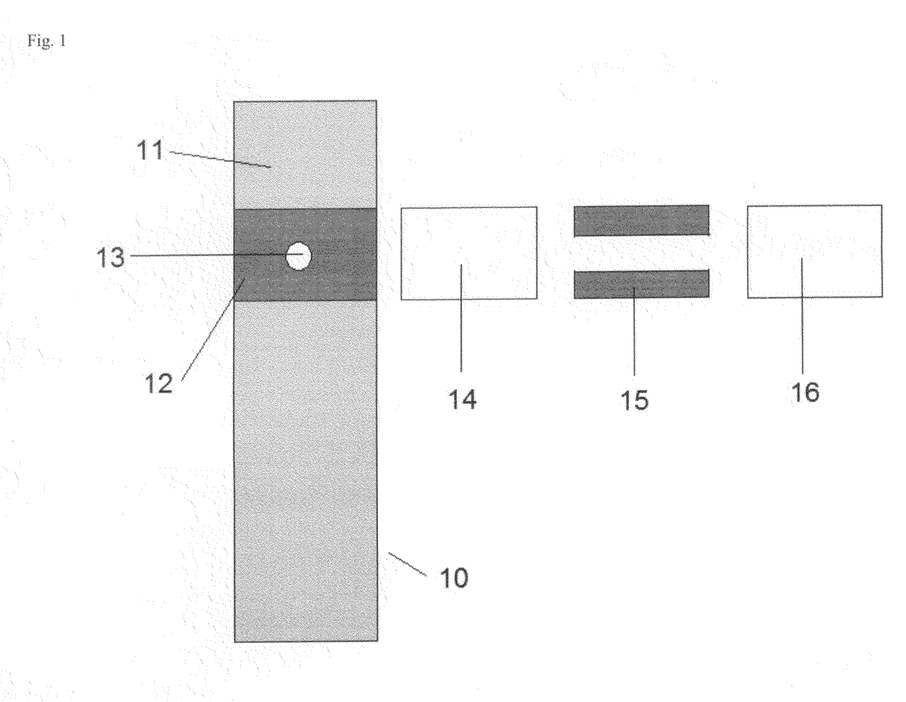

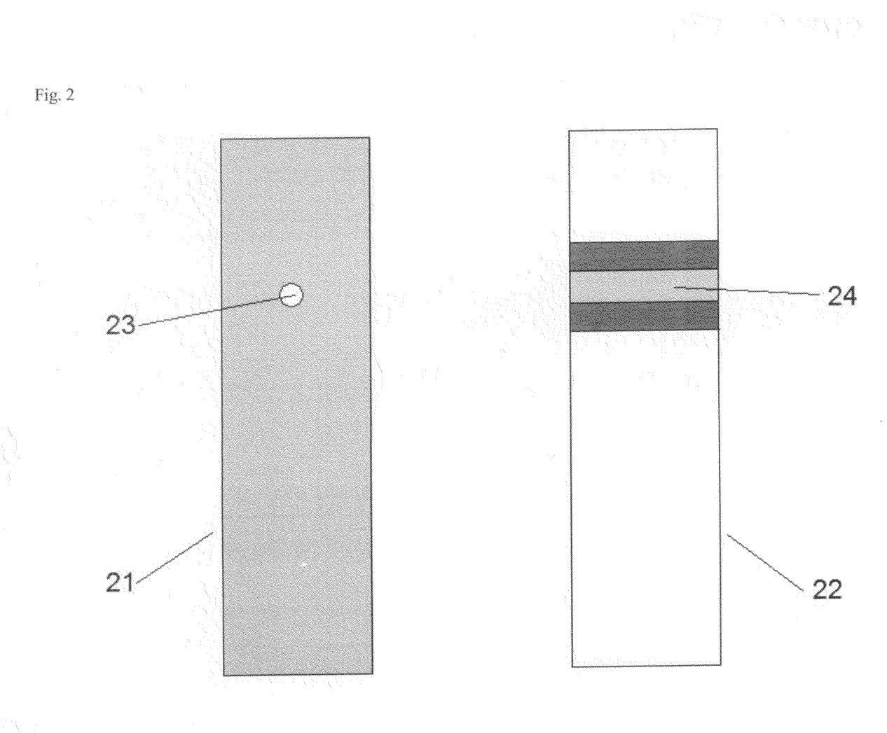

Unless otherwise indicated, all technical and scientific terms used herein have the same meaning as commonly understood by one of ordinary skill in the art. It is acknowledged that this invention is not limited to any particular embodiments described and that any embodiments similar or equivalent to those described herein can also be used in the practice or testing of the present invention. The ultra small volume and high speed optical sensors of the present invention are designed to measure the concentration of an analyte in a sample having a volume less than about 1 microliter, preferably less than about 0.5 microliter, more preferably less than about 0.3 microliter, and most preferably less than about 0.1 microliter, within 10 seconds of time, preferably within 5 seconds, and more preferably within 3 seconds. The analyte of interest is typically provided in a solution or biological fluid, such as blood or serum. As depicted in the Drawings in general and FIGS. 1 and 2 in particular, the optical sensors of the invention generally include a reaction membrane and a sample chamber. The sample chamber is constructed on one side of the membrane so that the other side is used for color detection.

### Reaction Membrane

The reaction membrane is a uniformly porous membrane impregnated with dried chemicals and enzymes required by the specific test of the interested analyte. Most commercially available hydrophilic membranes, including nylon, polyester and polysulfone, will work with the current invention. To prepare the test pad, a signal producing reagent solution is first formulated. The membrane is impregnated by this solution and then dried. Many signal generating systems can be used in the invention.

When oxidase/peroxidase enzymes are utilized, the following signal producing chemicals or chemical pairs can be used: 3-methyl-2-benzothiazolinone hydrazone (MBTH) and 3-dimethylaminobenzoic acid (DMAB) [U.S. Pat. No. 5,049,487 Phillips et al.], (MBTH) and 8-anilino-1-naphthalenessulfonate (ANS) [U.S. Pat. No. 5,453,360 Yu], MBTH and 3-dimethylaminobenzoic acid (DMAB) [U.S. Pat. No. 5,049,487 Phillips et al.], sulfonated-MBTH and N-(3-sulfopropyl)aniline (HALPS) [U.S. Pat. No. 4,396,714 Maeda et al.]. An example of end-point testing in a corresponding meter using N-ethyl-N-(2-hydroxy-3-sulfopropyl)-3,5-dimethylaniline (MAOS) and 4-Aminoantipyrine is disclosed in U.S. Pat. No. 6,040,195 to Carroll et al.

### Sensors

The optical sensors are constructed according to FIG. 1. Plastic support 11 is laminated with a double-sided adhesive 12. Detection hole 13 is punched through on the support and adhesive. Reaction membrane 14 is then laminated onto the plastic supports 11 by adhesive 12. Two pieces of adhesive 15 are laminated apart onto reaction membrane 14 and then covered by another piece of plastic film to create a sample dosage chamber.

FIG. 2 shows an example of a complete sensor. 21 is showing one side of the sensor with detection hole 23; and 22 is showing the other side of the sensor with sample dosage chamber 24.

### EXAMPLES

Following examples are used to further characterize the invention and not meant to limit the scope of the invention. Variations within the concepts of the invention are apparent to those skilled in the art.

### Example 1

### Preparation of Optical Sensor Membrane

A solution containing 40 mg MBTH, 80 mg DMAB, 10 mg EDTA disodium salt, 0.668 g sodium citrate, 0.523 g citric acid, 3000 units of horseradish peroxidase, 4500 units of glucose oxidase, 100 mg BSA and 100mg PVP-360K in 10 ml water was prepared. A piece of Pall Supor membrane, 0.8 micron, was coated with the solution. After blotting off excess liquid, the membrane was dried by forced airflow at room temperature within 15 minutes.

### Example 2

A reflectance sensor was constructed corresponding to the embodiment of the invention depicted in FIGS. 1 and 2.

A transparent styrene film of 0.13 mm thickness was laminated with a double-sided adhesive of 0.1 mm thickness and then punched with a hole of 1.5 mm diameter. The hole was covered with the sensor membrane prepared in Example 1. On the top of the membrane, two pieces of spacer adhesive of 0.1 mm were laminated to create the sample chamber of 1.5 mm width. A finished sensor is depicted in FIG. 2.

### Example 3

### Determination of Blood Glucose Concentration by Reflectance Sensor

A series of blood samples with different glucose concentrations were prepared and tested by the sensors.

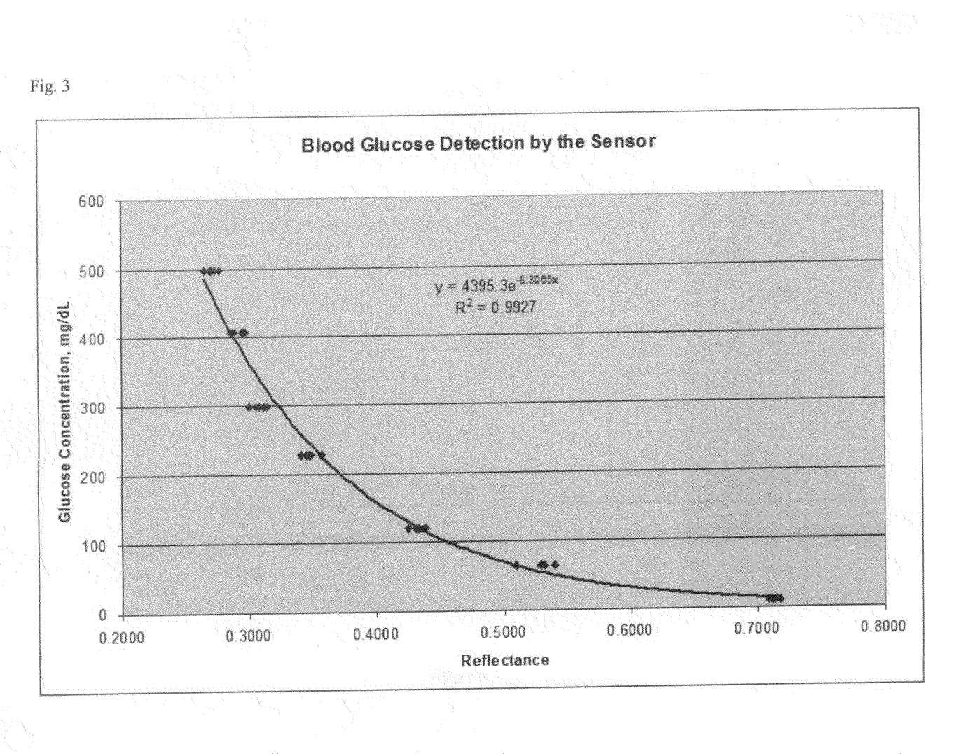

The results of the reflectance sensor are shown in Table 1 and in FIG. 3.

TABLE 1
Correlation between Glucose Concentrations and Reflectance Determined
by the Sensor at 3 Seconds of Reaction Time

| Blood Glucose | | | | |
|---|---|---|---|---|
| Concentration, | Reflectance at 3 | Average | Standard | |
| mg/dL | seconds | Reflectance | Deviation | CV, % |
| 11 | 0.7115 | | | |
| 11 | 0.7087 | | | |
| 11 | 0.7132 | | | |
| 11 | 0.7172 | | | |
| 11 | 0.7114 | 0.7124 | 0.0031 | 0.44% |
| 65 | 0.5285 | | | |
| 65 | 0.5092 | | | |
| 65 | 0.5400 | | | |
| 65 | 0.5289 | | | |
| 65 | 0.5313 | 0.5276 | 0.0113 | 2.14% |
| 119 | 0.4332 | | | |
| 119 | 0.4335 | | | |
| 119 | 0.4250 | | | |
| 119 | 0.4384 | | | |
| 119 | 0.4312 | 0.4323 | 0.0049 | 1.12% |
| 227 | 0.3574 | | | |
| 227 | 0.3421 | | | |
| 227 | 0.3491 | | | |
| 227 | 0.3462 | | | |
| 227 | 0.3482 | 0.3486 | 0.0056 | 1.61% |
| 299 | 0.3081 | | | |
| 299 | 0.3055 | | | |
| 299 | 0.2999 | | | |
| 299 | 0.3145 | | | |
| 299 | 0.3124 | 0.3081 | 0.0058 | 1.87% |
| 407 | 0.2881 | | | |
| 407 | 0.2862 | | | |
| 407 | 0.2941 | | | |
| 407 | 0.2976 | | | |
| 407 | 0.2951 | 0.2922 | 0.0048 | 1.66% |
| 498 | 0.2768 | | | |
| 498 | 0.2649 | | | |
| 498 | 0.2706 | | | |
| 498 | 0.2697 | | | |
| 498 | 0.2735 | 0.2711 | 0.0045 | 1.64% |

I claim:

1. A disposable sensor strip for measuring the amount of an analyte in a liquid sample, said sensor comprising:
(a) a base support member which provides support for said sensor and having an aperture for reaction inspection by a measurement instrument;
(b) a reaction membrane having a surface which is in direct physical contact with said support member and covers all of said aperture; and
(c) a capillary chamber for receiving and holding said sample, which capillary chamber encloses a capillary passage for conducting said sample from an incision to said capillary chamber and securing said sample over said reaction membrane, wherein:
(i) said capillary chamber is attached directly to said support member and said capillary passage is positioned directly over said reaction membrane;
(ii) the open ends of said capillary passage extends to the lateral edges of said support member;
(iii) the ventral side of said capillary passage is exposed entirely to said reaction membrane and the width of said capillary passage is no greater than the diameter of said aperture;
(iv) said capillary passage is configured to align said sample over an area of said reaction membrane in vertical alignment with said aperture; and
(v) said capillary passage is sized to contain a volume of less than about 1 μL of said sample.

2. The sensor of claim 1, wherein said sample passage is sized to contain a volume of less than about 0.5 μL of said sample.

3. The sensor of claim 2, wherein said sample passage is sized to contain a volume of less than about 0.1 μL of said sample.

4. The sensor of claim 1, wherein said aperture has a surface area no greater than approximately 1.0 mm².

5. The sensor of claim 1, wherein said support member comprises multiple indentations as means for engagement with said measurement instrument.

6. The sensor of claim 5, wherein said multiple indentations are bores with a surface area no greater than 1 mm² as means for engagement with said measurement instrument.

7. The sensor of claim 6, wherein upon engagement with said measurement instrument, said capillary chamber and said aperture both remain above said measurement instrument's light source.

8. The sensor of claim 1, wherein said reaction membrane comprises a hydrophilic material.

9. The sensor of claim 7, wherein said reaction membrane comprises at least one reagent for reacting with at least one compositional component of said sample.
